(19) United States
(12) Patent Application Publication
(10) Pub. No.: US 2024/0043812 A1
(43) Pub. Date: Feb. 8, 2024

(54) NON-INVASIVE METHOD FOR GENERATING HUMAN THREE-DIMENSIONAL AND TWO- DIMENSIONAL NASOPHARYNGEAL ORGANOIDS

(71) Applicant: Centre for Immunology & Infection Limited, Hong Kong (HK)

(72) Inventors: Chi Wai Michael CHAN, Hong Kong (HK); Pui Yan HUI, Hong Kong (HK); Hiu Ha CHING, Hong Kong (HK)

(21) Appl. No.: 18/359,916

(22) Filed: Jul. 27, 2023

Related U.S. Application Data
(60) Provisional application No. 63/395,316, filed on Aug. 4, 2022.

(51) Int. Cl. C12N 5/071

(52) U.S. Cl. CPC C12N 5/0688; C12N 2513/00; C12N 2501/155; C12N 2501/415; C12N 2501/119; C12N 2501/117; C12N 2509/00; C12N 2509/10; C12N 2503/00; C12N 2533/54

(57) ABSTRACT

The present invention relates to a non-invasive method for generating human three-dimensional nasopharyngeal organoids, including non-invasively collecting nasopharyngeal swab samples; culturing the samples in a matrix with niche factors; and obtaining the three-dimensional human nasopharyngeal organoids after culturing the samples in the matrix for 14-21 days. The three-dimensional human nasopharyngeal organoids are then dissociated into a single cell suspension for the creation of human two-dimensional nasopharyngeal organoids. The present nasopharyngeal organoids and their preparation method enable the rapid expansion of stem cells to form organoids within a short period of time. By combining organoid passaging techniques, a sufficient number of organoids can be obtained within a limited time for research and experimental operations. These organoid models provide new and excellent tools to study the transmission, tropism, and innate host responses of emerging viruses such as influenza virus and coronavirus, aiding in the evaluation of the pathophysiological characteristics of the viruses.

### CROSS-REFERENCE TO RELATED APPLICATION

The present application claims the priority from the U.S. provisional patent application Ser. No. 63/395,316 filed Aug. 4, 2022, and the disclosure of which is incorporated herein by reference in its entirety.

### FIELD OF THE INVENTION

The present invention generally relates to a method for obtaining human stem cells and generating human 3D and 2D nasopharyngeal (NP) organoids. More specifically, the present invention provides a non-invasive method for generating human NP organoids, which serve as a novel ex vivo model for studying the pathogenesis and tropism of emerging respiratory viruses, such as influenza viruses and coronaviruses.

### BACKGROUND OF THE INVENTION

The transmission and pathogenesis of respiratory viruses such as influenza virus and coronavirus are hitherto primarily studied using experimental animal models or in cell cultures in vitro. Mice have traditionally been used as a small mammalian model for the pathogenesis of influenza viruses and coronavirus (like severe acute respiratory syndrome coronavirus 2, SARS-CoV-2), but most human viruses do not adopt efficient replication in mice without prior adaptation. The absence of clinical signs comparable with human respiratory viruses in mice has also limited their utility as model for virus transmission.

Alternatively, ferrets are used as an attractive model for both pathogenesis and transmissibility of influenza and coronavirus infection. Human and avian influenza viruses and coronaviruses can replicate efficiently in the respiratory tract of ferrets without prior adaptation and infected ferrets have shown numerous clinical signs observed in humans following experimental virus inoculation. Successful virus transmission has been demonstrated between infected and virus-naïve ferrets through direct contact or respiratory droplets, thus the ferret represents an accurate model for human influenza disease and coronavirus infection. The ferret model is both expensive and has low throughput and the limitations in the numbers of animals that can be used in a given experiment makes it difficult to achieve robust data for statistical analysis. In vitro two-dimensional monolayer cell cultures of immortalized cell lines are valuable in studying cell biology and host regulation, but they fail to reconstitute the in vivo cellular microenvironment.

The use of adult stem cell or pluripotent stem cell derived organoids which have three-dimensional (3D) structures composed of differentiated cell types with tissue specific histology properties thus afford an interesting new addition to the spectrum of methods available for studying the pathogenesis and tropism of emerging respiratory viruses. Organoid culture has been reported to be able to retain its typical morphological characteristics and structure after long-term cryopreservation, making them a facile tool for clinical application. With well-differentiated human cell types available for viral infection, organoids faithfully replicate human pathology. Several studies have reported the generation of organoids representing the different regions of the lung including bronchiolar organoids, bronchioalveolar organoids and alveolar spheroids from human pluripotent stem cells. The resulting structure consists of multiple cell types found in that particular organ and the cells are able to execute functions similar to that in the native organ. Taken together with the “readily-available” and “ever-growing” properties of organoid model, these features make organoid as an attractive model for infectious diseases.

Although the ex vivo explant culture of the human NP tissue is so far the most physiological relevant human respiratory tract model for risk assessing the tropism of influenza virus and coronavirus and has now been validated as a parameter for risk assessment of animal influenza viruses for pandemic threat and is included as one parameter in the WHO “Tool for Influenza Pandemic Risk Assessment”, the availability of tissues from resected human NP is limited, the duration of viability in ex vivo is limited and it is difficult to control donor-to-donor variability.

Furthermore, the lack of a robust and reproducible in vitro culture model of the human respiratory epithelium hinders the understanding of the interactions between respiratory viruses and the host, as well as the pathology of the respiratory system. Establishment of adult stem-cell derived organoids relies on invasive sampling techniques to obtain the subject/patient samples.

Therefore, there is a need to develop more physiologically relevant in vitro epithelial infection models.

### SUMMARY OF THE INVENTION

Accordingly, the present invention describes a method for generating human

nasopharyngeal organoids, which provide a model that preserves epithelial cells in their native 3D environment. To facilitate the accessibility to various pathogens, these organoids can be converted into a 2D model, allowing easy access for pathogens to interact with the epithelium from both apical and basolateral chambers. Both 3D and 2D organoid models will be useful tools to study the transmission, tropism, and innate host responses of emerging viruses such as influenza virus and coronavirus. These models contribute to the evaluation of the pathophysiological characteristics of viruses.

In a first aspect, the present invention provides a non-invasive method for generating human nasopharyngeal (NP) organoids, which serves as a novel ex vivo model for studying the tropism and pathogenesis of newly emerging viruses. The method includes non-invasively collecting nasopharyngeal swab samples; culturing the nasopharyngeal swab samples in a matrix containing one or more niche factors for stimulating the three-dimensional nasopharyngeal organoids growth; obtaining one or more three-dimensional human nasopharyngeal organoids after culturing the nasopharyngeal swab samples in the matrix for 14-21 days.

In accordance with one embodiment, the three-dimensional nasopharyngeal organoids include a spherical structure with an average diameter between 20 μm and 100 μm.

In accordance with one embodiment, the niche factors comprise fibroblast growth factor, Wnt signal amplifier R-spondin and bone morphogenetic protein inhibitor Noggin.

In accordance with one embodiment, the one or more three-dimensional human nasopharyngeal organoids cultured on day 14 are passaged using an enzyme and/or mechanical shearing.

In accordance with one embodiment, the enzyme includes an animal origin-free, recombinant enzyme.

The animal origin-free, recombinant enzyme includes TrypLE™-Express enzyme. TrypLE is a specific trypsin-based dissociation reagent widely used in cell biology and cell culture experiments. TrypLE is a commercial trypsin reagent produced by Thermo Fisher Scientific. Compared to traditional trypsin, TrypLE offers several advantages: (1) Efficiency: TrypLE can digest protein linkages on the cell surface more rapidly and effectively, making cells easier to disperse and culture individually; (2) Stability: TrypLE exhibits higher stability in culture medium, minimizing the risk of inactivation or degradation; (3) Reproducibility: TrypLE has a tightly controlled composition, ensuring consistent enzyme activity and performance, thereby enhancing the reproducibility and reliability of experiments.

In accordance with one embodiment, the one or more three-dimensional human nasopharyngeal organoids are rich in p63a⁺ epithelial cells, SCGB1A1/CC10⁺ secretary club cells, acetyl-α-Tubulin⁺ ciliated cells and MUC5AC⁺ mucus secretary goblet cells.

In accordance with one embodiment, the three-dimensional human nasopharyngeal organoids contain four respiratory proximal epithelial cell types, including basal cells, club cells, ciliated cells and goblet cells.

In accordance with one embodiment, the human three-dimensional nasopharyngeal organoids serve as useful tools to study the transmission, tropism, and innate host responses of emerging respiratory viruses including influenza virus, which help to evaluate the pathophysiological characteristics of the emerging respiratory viruses.

In a second aspect of the present invention, one or more three-dimensional human nasopharyngeal organoids are obtained according to the first aspect. After that, the one or more three-dimensional human nasopharyngeal organoids are dissociated into a single cell suspension for the creation of one or more human two-dimensional nasopharyngeal organoid.

In accordance with one embodiment, one or more three-dimensional human nasopharyngeal organoids dissociated into a single cell suspension is further seeded in an insert pre-coated with rat tail collagen I until reaching a confluent monolayer. When reaching a confluent monolayer, the cells are differentiated for at least 8 days.

In accordance with one embodiment, the one or more two-dimensional human nasopharyngeal organoids contain four respiratory proximal epithelial cell types, including basal cells, club cells, ciliated cells and goblet cells. The ciliated cells are greatly increased in the two-dimensional human nasopharyngeal organoids. For example, the ciliated cells are increased by 24-fold in the two-dimensional human nasopharyngeal organoids when compared with that in the one or more three-dimensional human nasopharyngeal organoids.

In accordance with one embodiment, the human two-dimensional nasopharyngeal organoids serve as useful tools to study the transmission, tropism, and innate host responses of emerging respiratory viruses including coronavirus, which help to evaluate the pathophysiological characteristics of the emerging respiratory viruses.

The cellular morphology of NP organoids is physiologically similar to human nasopharynx, with cilia, club cells, goblet cells and basal cells tightly bound together by intercellular junctional complexes. The sialic acids (SAs) profile of the NP organoids showing a high expression of SAα2,3 Gal and SAα2,6Gal is similar to that of human nasopharynx. 2D differentiated NP organoids are disclosed which contain basal cells, club cells, goblets cells and enriched ciliated cells.

In accordance with another aspect of the present invention, the method includes assessing the infectivity, tropism and pathogenesis of newly emerging viruses, such as influenza viruses (H5N1 and H1N1 pandemic) and coronaviruses (SARS-CoV-2 variants) in 3D and 2D NP organoids.

### BRIEF DESCRIPTION OF THE DRAWINGS

Embodiments of the present invention are described in more detail hereinafter with reference to the drawings, in which:

FIG. 1 shows the procedure of human nasopharyngeal (NP) sample collection and 3D NP organoid formation. Nasopharyngeal (NP) swab samples were non-invasively collected by clinicians using a specially designed brush. The NP swab with cells was transferred to a tube containing 20 ml of ice-cold basal medium and kept on ice. Isolated cells were grown in suspension culture as clumps of cells in Matrigel with the supplement of a defined set of niche factors for stimulating 3D organoid growth, including fibroblast growth factor, Wnt signal amplifier R-spondin and bone morphogenetic protein inhibitor Noggin. Human NP organoids were obtained after culturing in Matrigel and a defined set of niche factors for 14-21 days;

FIG. 2 shows human 3D NP organoids. (A) Light microscopy image of Matrigel-embedded 3D organoid culture. (B) Representative H&E staining image of NP organoids illustrates a spherical structure composed of epithelial cells. Transmission electron microscope images of (C) basal cells with (D) tight junction;

FIG. 3 shows human 3D NP organoids containing multiple epithelial cell types. Fixed organoids were paraffin-embedded and sectioned prior to immunohistochemistry staining. Representative microscope images show NP organoids contained high abundant of (A) epithelial cells, (B) p63a⁺ basal cells and some (C) SCGB 1A1/CC10⁺ secretary club cells, a few of (D) acetyl-α-Tubulin⁺ ciliated cells and (E) MUC5AC⁺ goblet cell;

FIG. 4 shows the expression of lectin in human 3D NP organoids. Lectin binding to the NP organoids was performed using (A) SNA (SAα2,6Gαl) and (B) MMAI (SAα2,3 Gal);

FIG. 5 shows the viral replication kinetics for influenza viruses in human 3D NP organoids. (A) Graphs show the mean virus titer. The horizontal dotted line denotes the limit of detection in the TCID₅₀ assay. Error bars show SEM. (B) Graphs show the Area Under the Curve (24-72 h). Error bars show SEM. *p<0.05.TCID₅₀=median tissue culture infectious dose. AUC=Area Under the Curve;

FIG. 6 shows the cellular tropism of influenza virus in human 3D NP organoids. At 24 h post-infection the H1N1pdm- and H5N1-infected organoids were fixed in 4% Paraformaldehyde, embedded in paraffin and subjected to immunohistochemical (IHC) staining. Basal and club cells were stained with p63a and CC10, respectively. All sections were stained with a monoclonal antibody against the influenza nucleoprotein with positive cells identified as red/brown colour on immunohistochemical staining;

FIG. 7 shows cytokine and chemokine mRNA expression profile in human NP organoids infected with mock, H1N1pdm and H5N1 viruses. Expression of influenza matrix (M) gene, TNFα, IL6, IFNβ, RANTES/CCL5, and MCP-1/CCL2 24 h after infection. Graph shows mean mRNA copies expressed per 10⁵ β-actin copies; error bars show SEM. *p<0.05. **p<0.01. ***p<0.001;

FIG. 8 shows the percentages of individual cell types in 3D (top) and 2D (bottom) NP organoids as detected by FACS analysis;

FIG. 9 shows the viral replication kinetics of SARS-CoV-2 variants in Human 2D NP organoids. (A) Graphs show the mean virus titer. The horizontal dotted line denotes the limit of detection in the TCID₅₀ assay. (B) Graphs show the Area Under the Curve (24-48 h). TCID₅₀=median tissue culture infectious dose. AUC=Area Under the Curve; and

FIG. 10 shows the induction of interferon and cytokine responses by Omicron variants in human 2D NP organoids. The mRNA expression of the viral gene (ORF1b) of SARS-CoV-2, ACE2, IL29, IFNβ, IL6, IL8 and IP-10/CXCL10 after 48 h infection of SARS-CoV-2 variants were monitored using real-time PCR (polymerase chain reaction). Graph shows mean mRNA copies expressed per 10⁵ β-actin copies.

### DETAILED DESCRIPTION

The present invention will be described in detail through the following embodiments with appending drawings. It should be understood that the specific embodiments are provided for an illustrative purpose only, and should not be interpreted in a limiting manner. Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features. Other aspects and advantages of the invention will be apparent to those skilled in the art from a review of the ensuing description.

Limitations of ex-vivo cultures of human nasopharynx include the limited availability of such tissues, donor-to-donor variability in results, and the fact that these cultures are only viable for 48-72 h. Human NP organoids would be a valuable addition to such risk assessment algorithms because organoids from the same donor can be used repeatedly, can be efficiently retrieved from cryo-storage, and provide reproducible results in risk assessments done at different times, in different laboratories, with different viruses as they emerge, and will provide adequate replicate cultures to provide statistical robustness.

The transmission and pathogenesis of respiratory virus is hitherto primarily studied using experimental animal models or in cell cultures in vitro. Organoids, derived from stem cells or organ-specific progenitors, display structures and functions consistent with organs in vivo which can serve as a highly physiological relevant model to study the tropism and pathogenesis of respiratory viruses.

First, the present invention provides a non-invasive method for generating human nasopharyngeal (NP) organoids, which serves as a novel ex vivo model for studying the tropism and pathogenesis of newly emerging viruses. The method includes non-invasively collecting nasopharyngeal swab samples; culturing the nasopharyngeal swab samples in a matrix containing one or more niche factors for stimulating 3D organoid growth; obtaining one or more three-dimensional human nasopharyngeal organoids after culturing the nasopharyngeal swab samples in the matrix for 14-21 days.

The obtained nasopharyngeal organoids have a spherical structure. In one embodiment, the spherical structure has an average diameter between 20 μm and 100 μm.

Preferably, the spherical structure has an average diameter between 30 μm and 80 μm. For example, the spherical structure has an average diameter of 30 μm, 40 μm, 50 μm, 60 μm, 70 μm, or 80 μm.

Adding niche factors during the preparation of three-dimensional nasopharyngeal organoids is done to simulate the microenvironment within the nasopharynx, promoting the growth and functional differentiation of the organoids. These niche factors are molecular signaling substances that play a critical role in tissue and organ development. By adding niche factors during the culture of nasopharyngeal organoids, it is possible to mimic the cellular interactions and signaling within the nasopharynx, providing the necessary growth and differentiation signals for the organoids to better reflect the characteristics and functions of nasopharyngeal tissue.

In accordance with one embodiment, the niche factors may include, but are not limited, to fibroblast growth factor, Wnt signal amplifier R-spondin and bone morphogenetic protein inhibitor Noggin. These niche factors can include extracellular matrix components, cell-cell interaction molecules, growth factors, hormones, among others. By providing the appropriate nutrients and growth environment, niche factors can facilitate cell proliferation, differentiation, and the formation of tissue structures in nasopharyngeal organoids.

The 3D organoids cultured on day 14 are passaged using an animal origin-free, recombinant enzyme (TrypLE™-Express) and/or mechanical shearing. NP organoids are dissociated into cell aggregates/single cells using TrypLE™-Express (enzymatic dissociation method) for 5 min at 37° C., 100 rpm. The digest is sheared by pipetting the cell aggregate mixture up and down to break up the aggregates, spun at 400-500 g for 5 min at 4° C. The supernatant is removed, and the cell pellet is washed twice with ice-cold basal medium and spun at 400-500 g for 5 min at 4° C. After the last wash, the cell pellet is suspended in Matrigel and plated onto a 12-well plate. The plate is then incubated for 15 min at 37° C. Once the Matrigel domes are solidified, 1.5 ml of complete 3D NP organoid medium with 10 μM ROCK inhibitors (Y-27632) is added. ROCK inhibitors are added for the first 48 h after seeding to increase organoid formation efficiency and recovery from passaging.

TrypLE™-Express enzyme is a protease commonly used in cell culture for cell dissociation and cell cryopreservation processes. It is a cell dissociation enzyme developed by Thermo Fisher Scientific and is primarily used to separate cells from the culture surface for subsequent experimental or processing steps. TrypLE™-Express enzyme exhibits high activity and stability, allowing for quick and efficient cell dissociation without causing harm to the cells. This enzyme finds widespread application in cell biology and bioprocessing, particularly in cell culture, cell cryopreservation, and cell separation.

Additionally, the present invention further provides a non-invasive method for generating human two-dimensional nasopharyngeal organoids. The one or more three-dimensional human nasopharyngeal organoids are obtained according to the first aspect. After that, the one or more three-dimensional human nasopharyngeal organoids are dissociated into a single cell suspension for the creation of one or more human two-dimensional nasopharyngeal organoid.

In one embodiment, the cells in single cell suspension are further seeded in an insert pre-coated with rat tail collagen I until reaching a confluent monolayer. When reaching a confluent monolayer, the cells are differentiated for at least 8 days.

The use of nasopharyngeal (NP) organoid cultures derived from human upper lung stem cells, which have three-dimensional structures composed of differentiated cell types, tissue-specific histological properties, and sialic acid profiles similar to the human nasopharynx, provides an important addition to the experimental models available for the study of the tropism, replication kinetics, and host responses of influenza and coronavirus. Human NP organoids had similar morphological characteristics to human nasopharynx, including basal cells, club cells, goblet cells and ciliated epithelial cells. These organoids retain these features after expansion in in-vitro culture and after rescue from long-term cryopreservation making them a useful tool for research.

The present invention demonstrates that viral tropism and innate immune responses observed after infection of human NP organoids with human and avian influenza viruses, as well as coronavirus (SARS-CoV-2 variants), are comparable to those observed in ex-vivo explant models of the human conducting airway.

The following examples illustrate the present invention and are not intended to limit the same.

### EXAMPLE

### Example 1

### Methods and Sample Preparation

### Influenza Viruses and Coronaviruses

Highly pathogenic avian influenza virus (HPAI) H5N1 virus A/Hong Kong/483/1997 (483/H5N1), isolated from a fatal human infection; and a pandemic human H1N1 virus A/Hong Kong/415742/2009 (415742/H1N1pdm) isolated from a patient in Hong Kong were used. All influenza viruses were passaged in Madin-Darby Canine Kidney (MDCK). Vero E6 cells were used for virus isolation and propagation of the SARS-CoV-2 wild-type (WT) (hCoV-19/Hong Kong/WHV-HK61-P3/2020, Genbank accession ID: 0M403304) and Vero E6-TMPRSS2 overexpressed cells were used for the Omicron variants (BA.1: hCov-19/Hong Kong/V000195-P2/2021, Genbank accession ID: 0M403309; BA.2: SARS-CoV-2/human/HKG/Consensus_VOC-588-P3-S18-iseq/2022, Genbank accession ID: ON026861). Both cell lines were cultured in DMEM with 10% FBS.

Viruses were isolated from clinical samples of the nasopharyngeal and throat swab from patients infected with SARS-CoV-2 in virus transport medium. Vero E6-TMPRSS2 (E6/T2) cells seeded at 1×10⁵ cells in 24-well plate were inoculated with 50 μl of sample and topped up with 2% FBS DMEM medium to 1 ml for 1 h at 37° C. The cells were washed once with PBS, replenished with fresh medium and observed daily for cytopathic effect (CPE). Culture supernatants were collected when the CPE reached around 50% and was defined as P1. The virus was further propagated in E6/T2 cells. The virus stock was aliquoted and stored frozen at −80° C. Viral titers were determined by median tissue culture infectious dose (TCID₅₀) as described. All experiments were done in a biosafety level 3 facility.

### Human 3D and 2D Nasopharyngeal Organoid Isolation and Culture

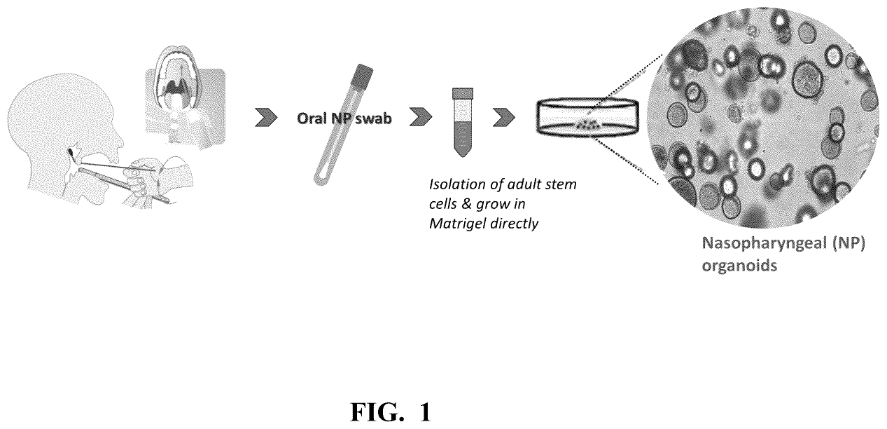

Nasopharyngeal (NP) swab samples were non-invasively collected by clinicians using a specially designed brush (FIG. 1). The upper airway stem cells collected from the NP swab were used to generate human 3D NP organoids. The NP swab with cells was transferred to a tube containing 20 ml of ice-cold basal medium and kept on ice. The tube containing brush was vortexed at a maximum speed to release cells. The suspension was transferred to a new 50 ml tube and centrifugated. Isolated cells were grown in suspension culture as clumps of cells in Matrigel with the supplement of a defined set of niche factors, including fibroblast growth factor, Wnt signal amplifier R-spondin and bone morphogenetic protein inhibitor Noggin.

Matrigel matrix (Growth Factor Reduced Basement Membrane Matrix; from Corning) is commonly used in cell culture and tissue engineering research, particularly when there is a need to provide an environment that allows cells to mimic physiological conditions or perform specific cellular function assays. It provides a platform that mimics the tissue environment in which cells reside. It is primarily composed of collagen, fibronectin, and other extracellular matrix molecules. Matrigel exhibits high biocompatibility and bioactivity, allowing it to simulate the structure and function of real tissues. Key features of Matrigel include: (1) Three-dimensional structure: Matrigel forms a three-dimensional mesh-like structure, providing the space required for cell attachment and growth; (2) Extracellular signaling: Matrigel contains various extracellular matrix molecules, such as extracellular matrix proteins, growth factors, and cell adhesion proteins, which can mimic the signals cells receive in the tissue; (3) Support of cellular function: Matrigel can influence cell proliferation, differentiation, and bioactivity, promoting the development of cell morphology and intercellular interactions.

The complete 3D NP organoid medium was prepared from basal medium (advanced DMEM/F12 supplemented with 1% GlutaMAX, 1% HEPES, 1% Penicillin/Streptomycin, 0.25 μg/ml Amphotericin B) supplemented with 10% R-spondin and 10% Noggin conditioned media, 25 ηg/ml FGF7, 200 ηg/ml FGF10, 0.5 μM A83-01, 10 μM SB202190, 1× B27, 1.25 mM N-Acetylcysteine, 10 mM Nicotinamide, 50 μg/ml Primocin. Human NP organoids were obtained after culturing in Matrigel and a defined set of niche factors for 14-21 days. The culture medium was changed every 2-3 days. For subculture, the 3D organoids cultured on day 14 are passaged using enzyme (TrypLE™-Express) and/or mechanical shearing.

To induce differentiated NP organoid monolayer (i.e. 2D NP organoid model), 3D organoids were digested using TrypLE™ Select (10×), sheared using 25G syringe and strained over a 40 μm cell strainer. Single cells (˜1.5e5) were seeded into each 24-well Transwell™ insert precoated with rat tail collagen type I. The cells were cultured in the expansion medium (in both apical and basal chambers) which was made by one part of complete 3D NP organoid medium and one part of PneumaCult™-ALI complete base medium (STEMCELL Technologies) for 2 to 3 days at 37° C. Once reaching a confluent monolayer, the expansion medium in both apical and basal chambers were gently aspirated and added Pneumacult-ALI medium (STEMCELL Technologies) supplemented with 10 μM DAPT (γ-secretase inhibitor) in basal chamber only (i.e. air-liquid interface culture) for at least 8 days to induce the differentiation process. Medium was replaced every 2-3 days.

### Respiratory Virus Infection of Human Nasopharyngeal Organoid

Human 3D nasopharyngeal organoids (approximately 80 μm in diameter) used for influenza virus infection were extracted from Matrigel droplets using Gentle Cell Dissociation Reagent (STEMCELL Technologies) and sheared by mechanical disruption using 1 ml pipettes to allow viruses to gain access to the apical and basolateral side of the epithelium. Around 100-200 organoids were infected with each influenza virus at 10⁶ TCID₅₀/mL for 1 h at 37° C. Organoids were washed with culture medium three times, re-embedded in Matrigel and plated 40-50 μl droplets per well in prewarmed (37° C.) 24-well suspension culture plates (Greiner). The culture plate was placed in a 37° C. incubator and incubated for about 15 minutes until the matrigel droplets were solidified.

Once solidified, the complete organoid medium was added (1 ml per well) and placed the 24-well plate in a 37° C. incubator with 5% CO₂. The viral titer in the culture supernatants was assessed at 1, 24, 48, and 72 hpi using the median tissue culture infectious dose (TCID₅₀) assay in MDCK cells. Cell lysates were collected 24 hpi to assess mRNA expression of cytokines using real-time polymerase chain reaction (RT-PCR) analysis.

Human 2D NP organoid models were used to assess the infectivity of SARS-CoV-2 variants. After washing the NP monolayers with basal medium, the cells were infected with the SARS-CoV-2 variants at a multiplicity of infection of 0.01 at the apical side (100 μ1) for 1 h at 33° C. Cells were washed with PBS twice and culture at ALI in the basal medium.

After 1, 24, 48 hours of infection, 200 μl of basal medium was added to the apical chamber of transwells and incubated it for 20 min at room temperature. All the supernatant in apical chamber was collected. The viral titer in the culture supernatants was assessed at 1, 24, 48 hpi using the median tissue culture infectious dose (TCID₅₀) assay in Vero-E6 or Vero E6-TMPRSS2 cells. Cell lysates were collected at 48 hpi for the mRNA expression using real-time polymerase chain reaction (RT-PCR) analysis.

### Viral Titration by TCID₅₀ Assay

A confluent 96-well tissue culture plate of MDCK or Vero-E6 or Vero E6-TMPRSS2 cells was prepared one day before the virus titration (TCID₅₀) assay. Cells were washed once with PBS and replenished with serum-free MEM medium (for MDCK cells) or DMEM with 2% fetal bovine serum (FBS) (for Vero-E6 and Vero E6-TMPRSS2 cells) supplemented with 1% penicillin/streptomycin. The MDCK cells were also treated with 2 μg/ml of TPCK (tosylsulfonyl phenylalanylchloromethyl ketone) treated trypsin. Serial dilutions of virus supernatant, from 0.5 log to 7 log, were performed before adding the virus dilutions onto the plates in quadruplicate. The plates were observed for cytopathic effect daily. The end-point of viral dilution leading to CPE in 50% of inoculated wells was estimated using the Karber method. AUC was calculated from viral titer from different time points indicated in the y-axis.

### Immunohistochemical Staining

Immunohistochemistry for several lung epithelial cell markers was carried out in the human nasopharyngeal organoids. The organoids were fixed in 4% paraformaldehyde solution (PFA), embedded and sliced into sections. The tissue sections were incubated with 0.05 mg/ml Pronase or microwaved for 15 min for antigen retrieval. Endogenous peroxidase activity was stopped by quenching the tissue sections with 3% H₂O₂ for 20 min. The slides were then blocked with 10% normal horse serum at room temperature (RT) and incubated with primary antibodies including Cytokeratin—AE1/AE3 (Dako), p63-alpha (Cell Signaling Technology), SCGB1A1/CC10 (Proteintech), acetyl-α-Tubulin (Santa Cruz Biotechnology), MUCSAC (Thermo Fisher Scientific) for 90 min at RT followed by peroxidase (HRP)-conjugated anti-rabbit or anti-mouse antibody (Vector Laboratory). The sections were developed using DAB/Vector®Red/NovaRED® Substrate Kit (Vector Laboratories). The cell nuclei were counterstained with Mayer' s Hematoxylin.

To characterize the infected cells, the infected organoids were fixed at 24 hpi. The double staining of SCGB1A1/CC10 (Proteintech) or p63-alpha (Cell Signaling Technology) with HB65 (EVL anti-influenza NP, subtype A) was performed. The tissue sections were first stained with HB65 similarly as mentioned above, except after NP antibody incubation, the sections were incubated with alkaline phosphatase (AP) or HRP conjugated anti-mouse antibody (Vector Laboratories) and developed using Vector® Red (VR) or DAB Substrate Kit (Vector Laboratories). The sections were then microwaved, incubated with p63-alpha or SCGB 1A1/CC10 (Proteintech) for 90 min at room temperature, followed by HRP or AP-conjugated anti-rabbit antibody (Vector Laboratories). The sections were developed using DAB or VR Substrate Kit (Vector Laboratories). The cell nuclei were counterstained with Mayer's Hematoxylin.

### Lectin Staining

Sialic acids (SAs) of cell surface glycoproteins and glycolipids are the receptors for the influenza virus.

Sialic acid distribution patterns on human airway organoids were studied

using lectin histochemical staining with biotinylated SNA, and MAAI from Vector Laboratories as described previously. Briefly, the paraffin-embedded tissues were sectioned, de-paraffinized, digested with Pronase and blocked with bovine serum albumin. The sections were then stained with biotinylated SNA or MAAI, followed by strepA-AP (Vector Laboratories). The sections were developed using VR-AP kit (Vector Laboratories) using Mayer's Hematoxylin to counterstain the nuclei.

### Transmission Electron Microscopy (TEM)

Organoids were fixed in 2.5% glutaraldehyde in cacodylate buffer (0.1 M sodium cacodylate-HCL buffered pH 7.4) for 1 h at 4° C., washed with cacodylate buffer with 0.1 M sucrose. The organoids were fixed in 1% osmium tetroxide (OsO4) in cacodylate buffer for 1 hour at RT, washed two times in cacodylate buffer, and resuspended in pre-warmed 2% agar and centrifuged at 3000 rpm for 10 min. The organoids were serially dehydrated with ethanol and propylene oxide. The organoids were embedded with epoxy resin. Sections were examined with a transmission electron microscope (Philips CM100).

### Real-Time PCR Assay

The RNA of infected cells was extracted at 24 or 48 hpi, using a MiniBEST universal RNA extraction kit (Takara). RNA was reverse-transcribed by using oligo-dT primers with PrimeScript™ RT reagent Kit (Takara). mRNA expression of target genes was performed using an ABI ViiA™ 7 real-time PCR system (Applied Biosystems). All procedures were performed according to the manufacturers' instructions. The gene expression profiles of cytokines and chemokines were quantified and normalized with β-actin.

### Flow Cytometry

The percentages of individual cell types in 3D and 2D NP organoids were detected by FACS analysis. Organoids were dissociated with TrypLE™ select (10×) for min at 37° C. to single cell suspension, fixed with 4% PFA and washed with PBS. The cells were permeabilized with 0.3% Triton-X100 and then incubated with primary antibodies including p63-alpha (Cell Signaling Technology), SCGB1A1/CC10 (R&D Systems), acetyl-α-Tubulin (Cell Signaling Technology), MUC5AC (Thermo Fisher Scientific) for 60 min at 4° C. followed by staining with Alexa Fluor™ 488-conjugated secondary antibodies. BD LSRFortessa™ cell analyzer was used to analyze the samples.

### Statistical Analysis

Statistical analysis was done using GraphPad Prism software version 9. Area Under the Curve (AUC) derived from viral titer was compared using unpaired t-test. mRNA expression was compared using one-way ANOVA with Bonferroni's multiple comparisons test. Mock infected organoids served as negative controls. Results shown in figures were the calculated mean and SEM. p<0.05 was considered to indicate a statistically significant difference.

### Example 2

### Characteristics of Human 3D Nasopharyngeal (NP) Organoids

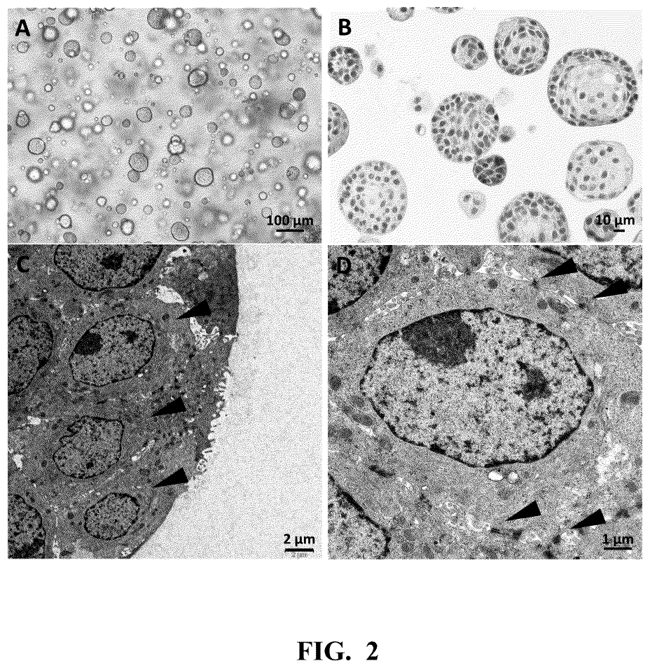

Human NP organoids with spherical structures (diameter 30-80 μm) were generated from the upper airway stem cells collected from NP swab (FIG. 2A). Histology analysis showed that the NP organoids exhibited a variety of epithelial cell types and accurately replicated the composition, cellular diversity, and organization observed in the human nasopharynx (FIG. 2B). The transmission electron microscope images showed the high expression of basal cells with tight junctions between cells of the NP organoids (FIGS. 2C, 2D).

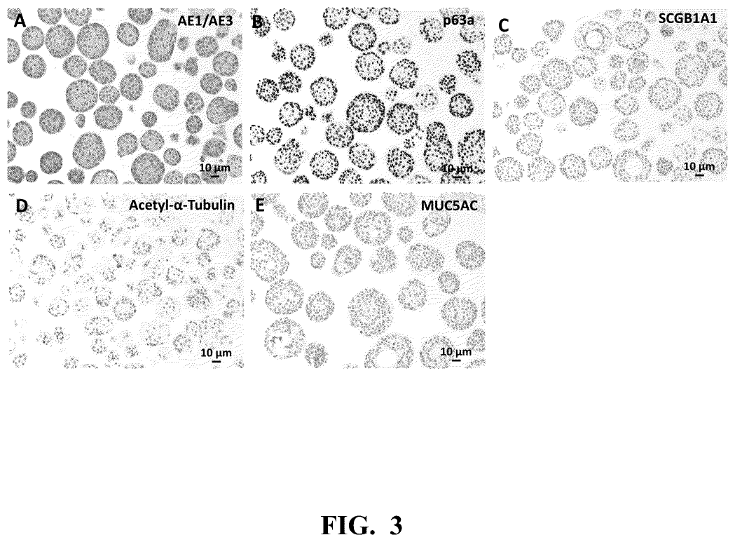

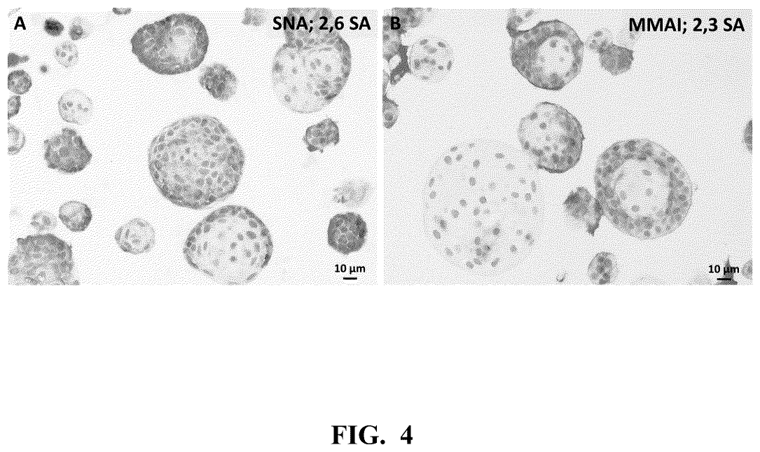

Immunohistochemical staining of the human NP organoids revealed that the organoid spheres were rich in epithelial cells (FIG. 3A), which contained high abundant of p63a+ basal cells (FIG. 3B), some SCGB 1A1/CC10+ secretary club cells (FIG. 3C), and a few of acetyl-α-Tubulin+ ciliated cells (FIG. 3D) and MUCSAC+ mucus secretary goblet cells (FIG. 3E), corresponding to the human nasopharyngeal mucosa. Lectin staining showed the NP organoids predominantly bound S nigra agglutinin (SNA) (FIG. 4A) and Maackia amurensis agglutinin 1 (MMAI) (FIG. 4B) and, indicating predominant expression of α-2,6-linked and α-2,3-linked sialic acids. It suggests that the NP organoid culture contains both human and avian sialic acid receptors.

### Example 3

### Replication Competence and Tissue Tropism of Influenza Viruses in Human 3D NP Organoids

The highly pathogenic avian influenza (HPAI) H5N1 virus is known to elicit robust proinflammatory cytokine responses that contribute to the pathogenesis of severe viral pneumonia and acute respiratory distress syndrome (ARDS). Compared with seasonal influenza, patients with H5N1 have increased serum concentrations of cytokines and chemokines, which contribute to pathogenesis, and this differential host response is also observed in cell cultures infected in vitro and in experimental animal models.

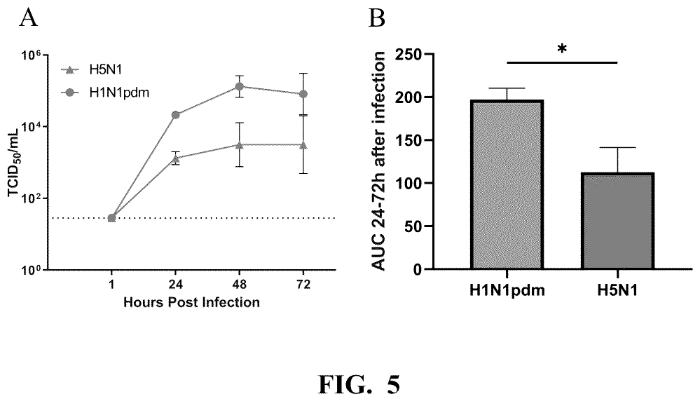

The replication kinetics of human 2009 pandemic H1N1pdm, HPAI H5N1 viruses in human 3D NP organoids were shown in FIG. 5A. Assessment of the Area Under the Curve integrated infectious virus titer 24-72 h after infection revealed that the replication of H1N1pdm was significantly higher than that of HPAI H5N1in NP organoid cultures (FIG. 5B).

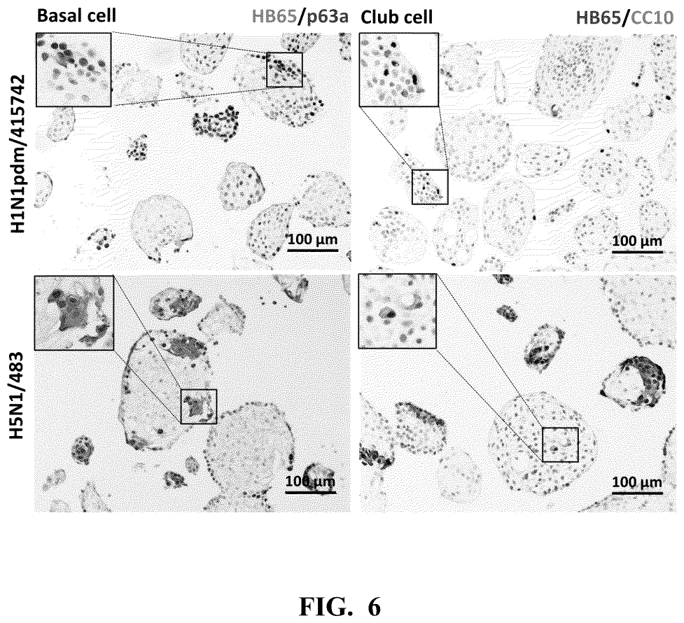

Immunohistochemical staining of human organoids revealed that H1N1pdm infected a higher number of cells when compared with HPAI H5N1, consistent with the viral replication kinetics of human NP organoids (FIG. 6). Both influenza viruses infected basal cells, but not club cells, conferring their role in viral entry and replication (FIG. 6).

H1N1pdm virus replicated more efficiently than H5N1 in the 3D NP organoids. This finding was similar to the observations in cultures of ex-vivo human upper respiratory tract in previous reports. The difference in virus replication kinetics observed in pandemic H1N1 and HPAI H5N1 was consistent with immunohistochemistry showing the number of virus-infected epithelial cells in the organoids. Basal cells were infected by the two influenza viruses in NP organoids, consistent with previous studies reporting that human and avian viruses bound to non-ciliated cells.

### Example 4

### Induction of Innate Immune System of Influenza A Viruses in Human 3D NP Organoid Cultures

The induction of proinflammatory cytokines, such as RANTES, is an important aspect of virulence. Although macrophages and dendritic cells are probably more important for the induction of cytokine responses, respiratory epithelial cells, which are the key target cells of influenza viruses, also contribute to host response cascades and pathogenesis. Therefore, the assessment of proinflammatory cytokines induction in airway epithelium is important for understanding how pathogenesis might differ between different strains of influenza A virus.

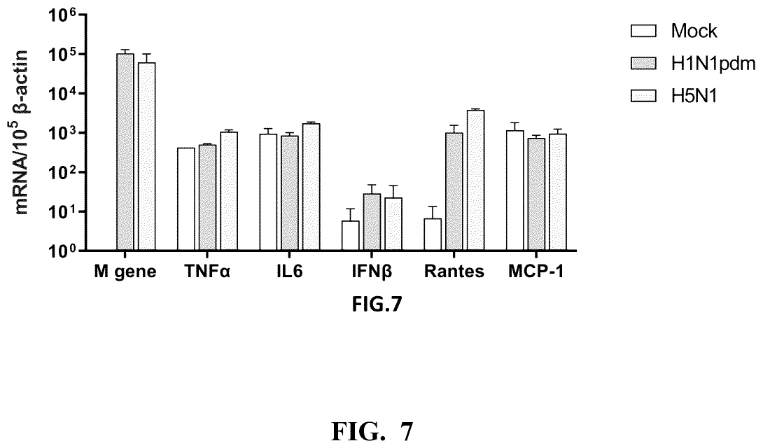

FIG. 7 showed that mRNA expression of influenza A infected NP organoids. M gene was comparable among the organoids infected with the two influenza viruses 24 hours post-infection. Nevertheless, organoids infected with HPAI H5N1 virus exhibited significantly higher expression of tumor necrosis factor alpha and RANTES compared to organoids infected with H1N1pdm virus.

### Example 5

### Characteristics of Human 2D Nasopharyngeal (NP) Organoids

To facilitate the accessibility of the apical surface to pathogens, the 3D

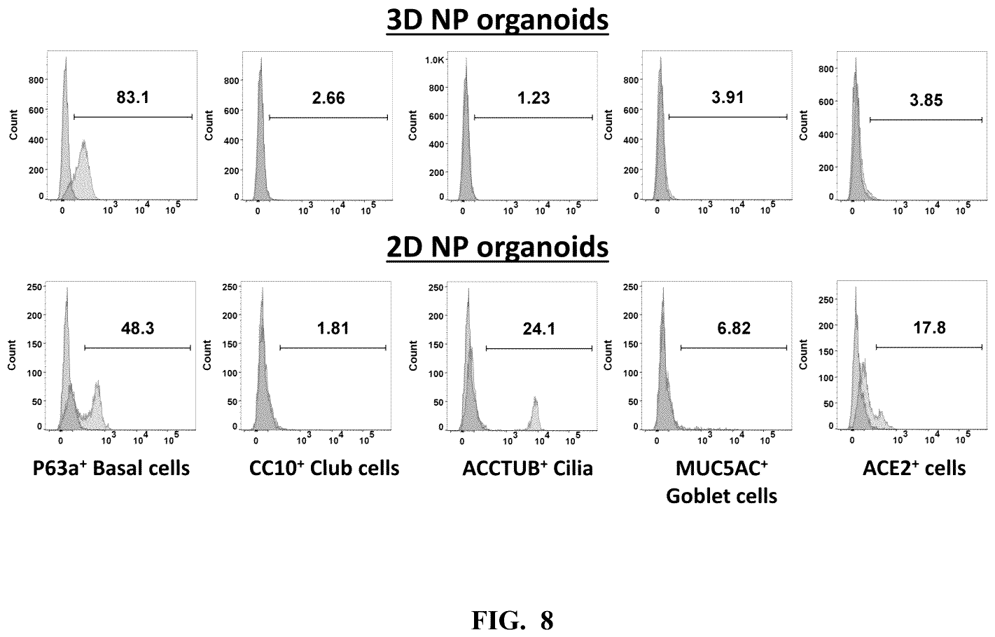

organoids were transformed into a 2D monolayer using Transwell™ culture. After 8-14 days of differentiation culture, 2D NP organoids with intact epithelium were successfully generated. The flow cytometry analysis was conducted to determine the percentages of four types of respiratory epithelial cells, it showed that both 3D and 2D NP organoids contained the four respiratory proximal epithelial cell types (basal cells, club cells, ciliated cells and goblet cells) where the basal cells and ciliated cells in 2D model were 2-fold decrease and 24-fold increase, respectively, when compared with that in 3D model (FIG. 8). The 2D differentiation process increased the expression of ACE2 identified as the entry receptor of SARS and SARS-CoV-2 (FIG. 8), which may serve as an optimized model to study infection of SARS-CoV-2 variants.

### Example 6

Replication competence of SARS-CoV-2 variants, and host innate immune and inflammatory responses to SARS-CoV-2 infection in human 2D NP organoids

The omicron variants of SARS-CoV-2 including BA.1 and BA.2 have been classified as a variant of concern (VOC), on the grounds of high transmission rate and rapid propagation in human populations, potential for immune evasion, unusual epidemiological properties, or adverse impact on diagnostics and therapeutics. Among Omicron, BA.2 is the dominant variant circulating in the world.

Omicron variants (BA.1 and BA.2) that were isolated from returning travelers or the community in Hong Kong in the 2D NP organoid platform by titrating infectious virus using TCID₅₀ titrations. The replication kinetics of SARS-CoV-2 wild type (WT),

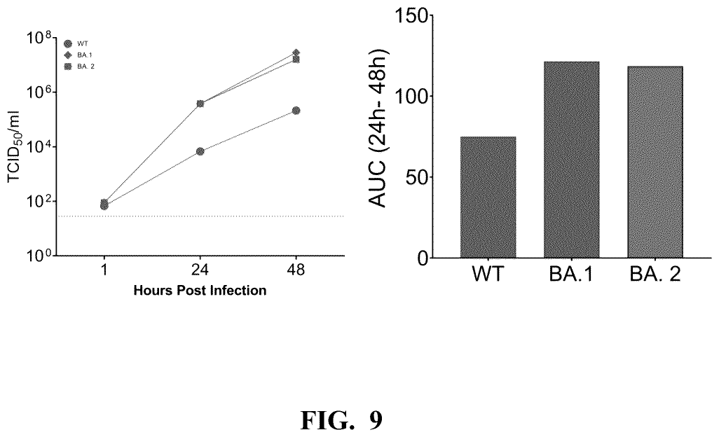

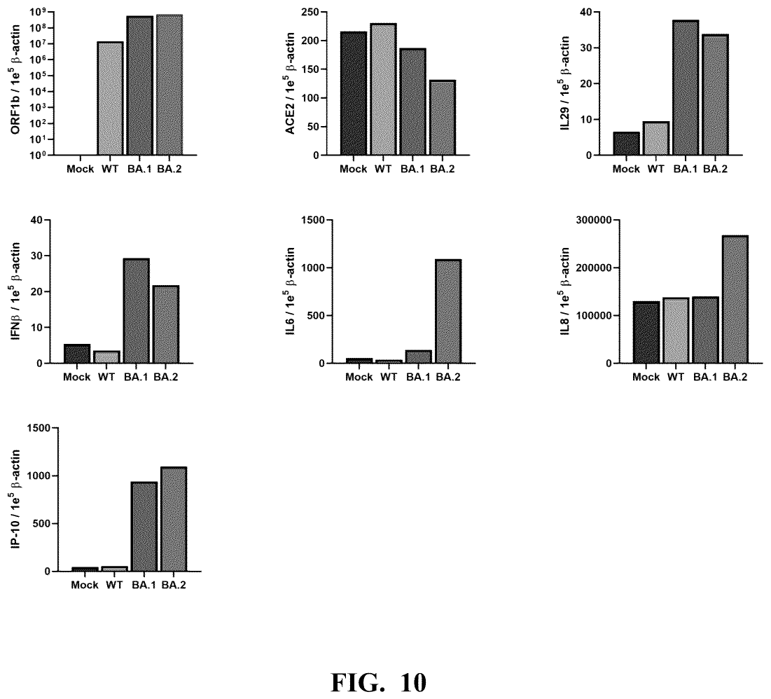

Omicron variants (BA.1 and BA.2) were shown in FIG. 9. Omicron variants (BA.1 and BA.2) replicated more efficiently than WT. FIG. 10 showed the cytokine profile of the 2D NP organoids infected with SARS-CoV-2 variants. The mRNA expression of ORFlb (viral gene) was higher in the NP organoids infected with BA.1 and BA.2 than that infected with WT. The expression of ACE2 was relatively lower in BA.2 infected NP organoids when compared with WT- and BA.1-infected organoids. Both BA.1 and BA.2 infection induced higher expression of IFNβ, IL29 (IFN-λ1) and IP-10 compared with WT infection. NP organoids infected by BA.2 greatly increased the expression of proinflammatory cytokines including IL6 and IL8.

In line with the studies, SARS-CoV-2 (WT) induced blunted IFN responses where the expression of IFNβ and IL29 (IFN-λ1) of the WT-infected NP organoids was comparable to that of uninfected organoids (mock). Whereas Omicron variants triggered host interferon response as indicated by the increased expression of IFNβ and IL29 in NP organoids infected with BA.1 and BA.2. This finding agrees with the observation previously reported that Omicron variants induced a stronger interferon response than the previous SARS-CoV-2 strains. As reported from Yamasoba et al (2022), compared with the original SARS-CoV-2 strain, the S protein of BA.2 bears>30 mutations, which may induce dramatically changes of the virological features of BA.2 from those of the original virus as well as the other variants.

Consistently, by using the 2D NP organoids, it shows that BA.2 may cause higher pathogenicity, as indicated by the greater expression of cytokines and chemokine IL6, IL8, IP-10 in BA.2 infected organoids compared with that infected with BA.1 and WT. Dramatic downregulation of ACE2 was observed in BA.2 infection, which may induce IL6 signaling leading to the hyper-inflammatory response.

In conclusion, the present invention demonstrates that influenza A viruses and coronaviruses can infect the NP organoids with similar replication competence and cellular localization to ex vivo human conducting airway explants, and thus provides an alternative physiologically relevant experimental platform for investigating virus tropism and replication competence that could be used to assess the pandemic threat of animal influenza viruses.

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The term “non-invasive” refers to a medical procedure, test, or treatment that does not require the insertion of instruments or devices into the body or the breaking of the skin. Non-invasive methods are designed to diagnose, monitor, or treat a condition without causing significant discomfort, pain, or trauma to the patient. The term “niche factor” can be used interchangeably herein with the term

“growth factor”. Using niche factors during cell growth in 3D nasopharyngeal (NP) organoids can be attributed to the following reasons:

(1) mimicking the natural environment: 3D NP organoids are in vitro tissue models designed to simulate the microenvironment of the nasopharyngeal tissue. By utilizing niche factors, it becomes possible to provide similar ecological factors present in the nasopharyngeal tissue, facilitating the proper development and functionality of the organoids.

(2) Supporting cell growth and differentiation: In the 3D NP organoid environment, niche factors can supply essential growth factors and extracellular matrix components to promote cell growth and differentiation. These factors mimic cell-cell communication and signaling present in the natural environment, ensuring the normal growth and development of cells within the organoids.

(3) Maintaining organoid structure and function: The use of niche factors helps maintain the structural integrity and specific tissue functions of 3D NP organoids. These factors can influence cell polarity, cell-cell connections, and cell-matrix interactions, aiding in the formation and preservation of tissue structure and facilitating functional performance of the organoids.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

It will be appreciated by those skilled in the art, in view of these teachings, that alternative embodiments may be implemented without undue experimentation or deviation from the spirit or scope of the invention, as set forth in the appended claims. This invention is to be limited only by the following claims, which include all such embodiments and modifications when viewed in conjunction with the above specification and accompanying drawings.

What is claimed is:

1. A non-invasive method for generating human three-dimensional nasopharyngeal organoids, comprising:
non-invasively collecting nasopharyngeal swab samples;
culturing the nasopharyngeal swab samples in a matrix containing one or more niche factors for stimulating the three-dimensional nasopharyngeal organoids growth; and
obtaining one or more three-dimensional human nasopharyngeal organoids after culturing the nasopharyngeal swab samples in the matrix for 14-21 days.

2. The method of claim 1, wherein the three-dimensional nasopharyngeal organoids comprise a spherical structure.

3. The method of claim 2, wherein the spherical structure has an average diameter between 20 μm and 100 μm.

4. The method of claim 1, wherein the niche factors comprise fibroblast growth factor, Wnt signal amplifier R-spondin and bone morphogenetic protein inhibitor Noggin.

5. The method of claim 1, wherein the one or more three-dimensional human nasopharyngeal organoids cultured on day 14 are passaged using an enzyme and/or mechanical shearing.

6. The method of claim 5, wherein the enzyme comprises an animal origin-free, recombinant enzyme.

7. The method of claim 1, wherein the one or more three-dimensional human nasopharyngeal organoids are rich in p63a⁺ epithelial cells, SCGB1A1/CC10⁺ secretary club cells, acetyl-α-Tubulin⁺ ciliated cells and MUC5AC⁺ mucus secretary goblet cells.

8. The method of claim 1, wherein the one or more three dimensional human nasopharyngeal organoids contain four respiratory proximal epithelial cell types.

9. The method of claim 8, wherein the four respiratory proximal epithelial cell types are basal cells, club cells, ciliated cells and goblet cells.

10. The method of claim 1, wherein the human three-dimensional nasopharyngeal organoids serve as useful tools to study the transmission, tropism, and innate host responses of emerging respiratory viruses comprising influenza virus, which help to evaluate the pathophysiological characteristics of the emerging respiratory viruses.

11. A non-invasive method for generating human two-dimensional nasopharyngeal organoids, comprising:
non-invasively collecting nasopharyngeal swab samples; culturing the nasopharyngeal swab samples in a matrix containing one or more niche factors for stimulating the three-dimensional nasopharyngeal organoids growth;
obtaining one or more three-dimensional human nasopharyngeal organoids after culturing the nasopharyngeal swab samples for 14-21 days; and
dissociating the one or more three-dimensional human nasopharyngeal organoids into a single cell suspension for the creation of one or more human two-dimensional nasopharyngeal organoid.

12. The method of claim 11, wherein the niche factors comprise fibroblast growth factor, Wnt signal amplifier R-spondin and bone morphogenetic protein inhibitor Noggin.

13. The method of claim 11, wherein the one or more three-dimensional human nasopharyngeal organoids cultured on day 14 are passaged using an enzyme and/or mechanical shearing.

14. The method of claim 13, wherein the enzyme comprises an animal origin-free, recombinant enzyme.

15. The method of claim 11, wherein one or more three-dimensional human nasopharyngeal organoids dissociated into a single cell suspension is further seeded in an insert pre-coated with rat tail collagen I until reaching a confluent monolayer.

16. The method of claim 15, when reaching a confluent monolayer, the cells are differentiated for at least 8 days.

17. The method of claim 11, wherein the one or more two-dimensional human nasopharyngeal organoids contain four respiratory proximal epithelial cell types.

18. The method of claim 17, wherein the four respiratory proximal epithelial cell types are basal cells, club cells, ciliated cells and goblet cells, and wherein the ciliated cells are increased by 24-fold in the two-dimensional human nasopharyngeal organoids when compared with that in the one or more three-dimensional human nasopharyngeal organoids.

19. The method of claim 11, wherein the human two-dimensional nasopharyngeal organoids serve as useful tools to study the transmission, tropism, and innate host responses of emerging respiratory viruses comprising coronavirus, which help to evaluate the pathophysiological characteristics of the emerging respiratory viruses.
